(19) United States
(12) Patent Application Publication
(10) Pub. No.: US 2023/0364131 A1
(43) Pub. Date: Nov. 16, 2023

(54) GOLD CLUSTERS, COMPOSITIONS, AND METHODS FOR TREATMENT OF CEREBRAL ISCHEMIC STROKES

(71) Applicant: SHENZHEN PROFOUND VIEW PHARMACEUTICAL TECHNOLOGY CO., LTD., SHENZHEN (CN)

(72) Inventor: Taolei SUN, Wuhan (CN)

(73) Assignee: SHENZHEN PROFOUND VIEW PHARMACEUTICAL TECHNOLOGY CO., LTD., SHENZHEN, GD (CN)

(21) Appl. No.: 18/249,253

(22) PCT Filed: Nov. 27, 2020
(86) PCT No.: PCT/CN2020/132280

§ 371 Date: Apr. 18, 2023

(51) Int. Cl. A61K 33/242; A61K 47/62; A61P 9/04; A61K 9/51

(52) U.S. Cl. CPC A61K 33/242; A61K 47/62; A61P 9/04; A61K 9/51

(57) ABSTRACT

Ligand-bound gold clusters and compositions comprising the ligand-bound gold clusters are used for treating cerebral ischemic stroke and manufacturing a medicament for treatment of cerebral ischemic stroke. Methods for treating cerebral ischemic stroke.

### FIELD OF THE INVENTION

The present invention relates to the technical field of brain illness treatment, particularly to ligand-bound gold clusters (AuCs), composition comprising the ligand-bound AuCs, use of the ligand-bound AuCs to prepare medications for treatment of cerebral ischemic stroke, and methods employing the ligand-bound AuCs and composition for treatment of cerebral ischemic stroke.

### BACKGROUND OF THE INVENTION

A stroke occurs when a blood vessel is either blocked by a clot or encountered ruptures. There are three types of stroke, i.e. cerebral hemorrhagic stroke, cerebral ischemic stroke, and transient ischemic attack (TIA).

Cerebral hemorrhagic stroke is caused by a blood vessel rupturing and preventing blood flow to the brain. The common symptoms include sudden weakness, paralysis in any part of the body, inability to speak, vomiting, difficulty walking, coma, loss of consciousness, stiff neck and dizziness. No specific medication is available.

Cerebral ischemic stroke, also known as brain ischemia and cerebral ischemia, represents one of the most prevalent pathologies in humans and is a leading cause of death and disability. Cerebral ischemic stroke is accounting for approximately 87 percent of all strokes. Cerebral ischemic stroke is caused by a blockage such as a blood clot or plaque in an artery that supplies blood to the brain, where the blockage appears at the neck or in the skull, and reduces the blood flow and oxygen to the brain, leading to damage or death of brain cells. If blood circulation is not restored quickly, brain damage can be permanent.

Specific symptoms of a cerebral ischemic stroke depend on what region of the brain is affected. Common symptoms for most ischemic stroke include vision problems, weakness or paralysis in limbs, dizziness and vertigo, confusion, loss of coordination, and drooping of face on one side. Once symptoms start, it is crucial to get treatment as quickly as possible, making it less likely that damage becomes permanent.

The main treatment for cerebral ischemic stroke is intravenous tissue plasminogen activator (tPA) that breaks up clots. The tPA has to be given within four and a half hours from the start of a stroke to be effective. However, tPA causes bleeding so that patients cannot be treated with tPA if they have a history of hemorrhagic stroke, bleeding in the brain, and recent major surgery or head injury. Long-term treatments include aspirin or an anticoagulant to prevent further clots.

Amani et al. disclose that OX26@GNPs formed by conjugating of OX26-PEG to the surface of 25 nm colloidal gold nanoparticles significantly increased the infarcted brain tissue, and bare GNPs and PEGylated GNPs had no effect on the infarct volume; their results showed that OX26@GNPs are not suitable for treatment of ischemic stroke.

Zheng et al. disclose that in their OGD/R injury rat model, 20 nm Au-NPs increased cell viability, alleviated neuronal apoptosis and oxidative stress, and improved mitochondrial respiration. However, Zheng et al. also demonstrated that 5 nm Au NPs showed opposite effects, not suitable for treatment of ischemic stroke.

TIA is caused by a temporary clot. The common symptoms include weakness, numbness or paralysis on one side of the body, slurred or garbled speech, blindness, and vertigo. No specific medication is available.

There remains a need for effective method and medications for treatment of cerebral ischemic stroke.

### SUMMARY OF THE INVENTION

The present invention provides the use of ligand-bound gold clusters to treat the cerebral ischemic stroke in a subject, the method of treating the cerebral ischemic stroke in a subject with ligand-bound gold clusters, and the use of ligand-bound gold clusters for manufacture of medicament for treatment of the cerebral ischemic stroke in a subject.

Certain embodiments of the present invention use of a ligand-bound gold cluster to treat the cerebral ischemic stroke in a subject, wherein the ligand-bound gold cluster comprises a gold core; and a ligand bound to the gold core.

In certain embodiments of the treatment use, the gold core has a diameter in the range of 0.5-3 nm. In certain embodiments, the gold core has a diameter in the range of 0.5-2.6 nm.

In certain embodiments of the treatment use, the ligand is one selected from the group consisting of L-cysteine and its derivatives, D-cysteine and its derivatives, cysteine-containing oligopeptides and their derivatives, and other thiol-containing compounds.

In certain embodiments of the treatment use, the L-cysteine and its derivatives are selected from the group consisting of L-cysteine, N-isobutyryl-L-cysteine (L-NIBC), and N-acetyl-L-cysteine (L-NAC), and the D-cysteine and its derivatives are selected from the group consisting of D-cysteine, N-isobutyryl-D-cysteine (D-NIBC), and N-acetyl-D-cysteine (D-NAC).

In certain embodiments of the treatment use, the cysteine-containing oligopeptides and their derivatives are cysteine-containing dipeptides, wherein the cysteine-containing dipeptides are selected from the group consisting of L(D)-cysteine-L(D)-arginine dipeptide (CR), L(D)-arginine-L(D)-cysteine dipeptide (RC), L(D)-histidine-L(D)-cysteine dipeptide (HC), and L(D)-cysteine-L(D)-histidine dipeptide (CH).

In certain embodiments of the treatment use, the cysteine-containing oligopeptides and their derivatives are cysteine-containing tripeptides, wherein the cysteine-containing tripeptides are selected from the group consisting of glycine-L(D)-cysteine-L(D)-arginine tripeptide (GCR), L(D)-proline-L(D)-cysteine-L(D)-arginine tripeptide (PCR), L(D)-lysine-L(D)-cysteine-L(D)-proline tripeptide (KCP), and L(D)-glutathione (GSH).

In certain embodiments of the treatment use, the cysteine-containing oligopeptides and their derivatives are cysteine-containing tetrapeptides, wherein the cysteine-containing tetrapeptides are selected from the group consisting of glycine-L(D)-serine-L(D)-cysteine-L(D)-arginine tetrapeptide (GSCR), and glycine-L(D)-cysteine-L(D)-serine-L(D)-arginine tetrapeptide (GCSR).

In certain embodiments of the treatment use, the cysteine-containing oligopeptides and their derivatives are cysteine-containing pentapeptide, wherein the cysteine-containing pentapeptides are selected from the group consisting of Cysteine-Aspartic acid-Glutamic acid-Valine-Aspartic acid (CDEVD) and Aspartic acid-Glutamic acid-Valine-Aspartic acid -Cysteine (DEVDC).

In certain embodiments of the treatment use, the other thiol-containing compounds are selected from the group consisting of 1-[(2S)-2-methyl-3-thiol-1-oxopropyl]-L(D)-proline, thioglycollic acid, mercaptoethanol, thiophenol, D-3-trolovol, N-(2-mercaptopropionyl)-glycine, dodecyl mercaptan, 2-aminoethanethiol (CSH), 3-mercaptopropionic acid (MPA), and 4-mercaptobenoic acid (p-MBA).

Certain embodiments of the present invention use a ligand-bound gold cluster for manufacture of a medicament for the treatment of the cerebral ischemic stroke in a subject, wherein the ligand-bound gold cluster comprises a gold core; and a ligand bound the gold core.

In certain embodiments of the manufacture use, the gold core has a diameter in the range of 0.5-3 nm. In certain embodiments, the gold core has a diameter in the range of 0.5-2.6 nm.

In certain embodiments of the manufacture use, the ligand is one selected from the group consisting of L-cysteine and its derivatives, D-cysteine and its derivatives, cysteine-containing oligopeptides and their derivatives, and other thiol-containing compounds.

In certain embodiments of the manufacture use, the L-cysteine and its derivatives are selected from the group consisting of L-cysteine, N-isobutyryl-L-cysteine (L-NIBC), and N-acetyl-L-cysteine (L-NAC), and the D-cysteine and its derivatives are selected from the group consisting of D-cysteine, N-isobutyryl-D-cysteine (D-NIBC), and N-acetyl-D-cysteine (D-NAC).

In certain embodiments of the manufacture use, the cysteine-containing oligopeptides and their derivatives are cysteine-containing dipeptides, wherein the cysteine-containing dipeptides are selected from the group consisting of L(D)-cysteine-L(D)-arginine dipeptide (CR), L(D)-arginine-L(D)-cysteine dipeptide (RC), L(D)-histidine-L(D)-cysteine dipeptide (HC), and L(D)-cysteine-L(D)-histidine dipeptide (CH).

In certain embodiments of the manufacture use, the cysteine-containing oligopeptides and their derivatives are cysteine-containing tripeptides, wherein the cysteine-containing tripeptides are selected from the group consisting of glycine-L(D)-cysteine-L(D)-arginine tripeptide (GCR), L(D)-proline-L(D)-cysteine-L(D)-arginine tripeptide (PCR), L(D)-lysine-L(D)-cysteine-L(D)-proline tripeptide (KCP), and L(D)-glutathione (GSH).

In certain embodiments of the manufacture use, the cysteine-containing oligopeptides and their derivatives are cysteine-containing tetrapeptides, wherein the cysteine-containing tetrapeptides are selected from the group consisting of glycine-L(D)-serine-L(D)-cysteine-L(D)-arginine tetrapeptide (GSCR), and glycine-L(D)-cysteine-L(D)-serine-L(D)-arginine tetrapeptide (GCSR).

In certain embodiments of the manufacture use, the cysteine-containing oligopeptides and their derivatives are cysteine-containing pentapeptide, wherein the cysteine-containing pentapeptides are selected from the group consisting of Cysteine-Aspartic acid-Glutamic acid-Valine-Aspartic acid (CDEVD) and Aspartic acid-Glutamic acid-Valine-Aspartic acid-Cysteine (DEVDC).

In certain embodiments of the manufacture use, the other thiol-containing compounds are selected from the group consisting of 1-[(2S)-2-methyl-3-thiol-1-oxopropyl]-L(D)-proline, thioglycollic acid, mercaptoethanol, thiophenol, D-3-trolovol, N-(2-mercaptopropionyl)-glycine, dodecyl mercaptan, 2-aminoethanethiol (CSH), 3-mercaptopropionic acid (MPA), and 4-mercaptobenoic acid (p-MBA).

The objectives and advantages of the invention will become apparent from the following detailed description of preferred embodiments thereof in connection with the accompanying drawings.

### DESCRIPTION OF THE DRAWINGS

Preferred embodiments according to the present invention will now be described with reference to the Figures, in which like reference numerals denote like elements.

FIG. 1 shows ultraviolet-visible (UV) spectrums, transmission electron microscope (TEM) images and particle size distribution diagrams of ligand L-NIBC-modified gold nanoparticles (L-NIBC-AuNPs) with different particle sizes.

FIG. 2 shows ultraviolet-visible (UV) spectrums, TEM images and particle size distribution diagrams of ligand L-NIBC-bound gold clusters (L-NIBC-AuCs) with different particle sizes.

FIG. 3 shows infrared spectra of L-NIBC-AuCs with different particle sizes.

FIG. 4 shows UV, infrared, TEM, and particle size distribution diagrams of ligand CR-bound gold clusters (CR-AuCs).

FIG. 5 shows UV, infrared, TEM, and particle size distribution diagrams of ligand RC-bound gold clusters (RC-AuCs).

FIG. 6 shows UV, infrared, TEM, and particle size distribution diagrams of ligand 1-[(2S)-2-methyl-3-thiol-1-oxopropyl]-L-proline (i.e., Cap)-bound gold clusters (Cap-AuCs).

FIG. 7 shows UV, infrared, TEM, and particle size distribution diagrams of ligand GSH-bound gold clusters (GSH-AuCs).

FIG. 8 shows UV, infrared, TEM, and particle size distribution diagrams of ligand D-NIBC-bound gold clusters (D-NIBC-AuCs).

FIG. 9 shows UV, infrared, TEM, and particle size distribution diagrams of ligand L-cysteine-bound gold clusters (L-Cys-AuCs).

FIG. 10 shows UV, infrared, TEM, and particle size distribution diagrams of ligand 2-aminoethanethiol-bound gold clusters (CSH-AuCs).

FIG. 11 shows UV, infrared, TEM, and particle size distribution diagrams of ligand 3-mercaptopropionic acid-bound gold clusters (MPA-AuCs).

FIG. 12 shows UV, infrared, TEM, and particle size distribution diagrams of ligand 4-mercaptobenoic acid-bound gold clusters (p-MBA-AuCs).

FIG. 13 shows UV, TEM, and particle size distribution diagrams of ligand 4-Cysteine-Aspartic acid-Glutamic acid-Valine-Aspartic acid (CDEVD)-bound gold clusters (CDEVD-AuCs).

FIG. 14 shows UV, TEM, and particle size distribution diagrams of ligand 4-Aspartic acid-Glutamic acid-Valine-Aspartic acid-Cysteine (DEVDC)-bound gold clusters (DEVDC-AuCs).

FIG. 15 shows the neurological behavior scores of rats in each group (in the histogram of each time point, from left to right are sham operation group (blank), model control group, A1 low-dose group, A1 high-dose group, A2 low-dose group, A2 high-dose group, A3 low-dose group, A3 high-dose group, A4 low-dose group, A4 high-dose group, B1 low-dose group, B1 high-dose group, B2 low-dose group and B2 high-dose group).

FIG. 16 shows the percentage of cerebral infarction area of rats in each group (in the histogram, from left to right are sham operation group (blank), model control group, A1 low-dose group, A1 high-dose group, A2 low-dose group, A2 high-dose group, A3 low-dose group, A3 high-dose group, A4 low-dose group, A4 high-dose group, B1 low-dose group, B1 high-dose group, B2 low-dose group and B2 high-dose group).

FIG. 17 shows the exemplary TTC staining images of brain tissues in MCAO rats after administration of gold cluster drugs and gold nanoparticles, where, (1) sham operation group; (2) model control group; (3) A1 low-dose group; (4) A1 high-dose group; (5) B1 low-dose group; (6) B1 high-dose group.

### DETAILED DESCRIPTION OF THE EMBODIMENTS

The present invention may be understood more readily by reference to the following detailed description of certain embodiments of the invention.

Throughout this application, where publications are referenced, the disclosures of these publications are hereby incorporated by reference, in their entireties, into this application in order to more fully describe the state of art to which this invention pertains.

As used herein, “administering” means oral (“po”) administration, administration as a suppository, topical contact, intravenous (“iv”), intraperitoneal (“ip”), intramuscular (“im”), intralesional, intrahippocampal, intracerebroventricular, intranasal or subcutaneous (“sc”) administration, or the implantation of a slow-release device e.g., a mini-osmotic pump or erodible implant, to a subject. Administration is by any route including parenteral and transmucosal (e.g., oral, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The terms “systemic administration” and “systemically administered” refer to a method of administering a compound or composition to a mammal so that the compound or composition is delivered to sites in the body, including the targeted site of pharmaceutical action, via the circulatory system. Systemic administration includes, but is not limited to, oral, intranasal, rectal and parenteral (i.e. other than through the alimentary tract, such as intramuscular, intravenous, intra-arterial, transdermal and subcutaneous) administration, with the proviso that, as used herein, systemic administration does not include direct administration to the brain region by means other than via the circulatory system, such as intrathecal injection and intracranial administration.

As used herein, the terms “treating” and “treatment” refer to delaying the onset of, retarding or reversing the progress of, or alleviating or preventing either the disease or condition to which the term applies, or one or more symptoms of such disease or condition.

The terms “patient,” “subject” or “individual” interchangeably refers to a mammal, for example, a human or a non-human mammal, including primates (e.g., macaque, pan troglodyte, pongo), a domesticated mammal (e.g., felines, canines), an agricultural mammal (e.g., bovine, ovine, porcine, equine) and a laboratory mammal or rodent (e.g., rattus, murine, lagomorpha, hamster, guinea pig).

Gold clusters (AuCs) are a special form of gold existing between gold atoms and gold nanoparticles. AuCs have a size smaller than 3 nm, and are composed of only several to a few hundreds of gold atoms, leading to the collapse of face-centered cubic stacking structure of gold nanoparticles. As a result, AuCs exhibit molecule-like discrete electronic structures with distinct HOMO-LUMO gap unlike the continuous or quasi-continuous energy levels of gold nanoparticles. This leads to the disappearance of surface plasmon resonance effect and the corresponding plasmon resonance absorption band (520±20 nm) at UV-Vis spectrum that possessed by conventional gold nanoparticles.

The present invention provides a ligand-bound AuC.

In certain embodiments, the ligand-bound AuC comprises a ligand and a gold core, wherein the ligand is bound to the gold core. The binding of ligands with gold cores means that ligands form stable-in-solution complexes with gold cores through covalent bond, hydrogen bond, electrostatic force, hydrophobic force, van der Waals force, etc In certain embodiments, the diameter of the gold core is in the range of 0.5-3 nm. In certain embodiments, the diameter of the gold core is in the range of 0.5-2.6 nm.

In certain embodiments, the ligand of the ligand-bound AuC is a thiol-containing compound or oligopeptide. In certain embodiments, the ligand bonds to the gold core to form a ligand-bonded AuC via Au—S bond.

In certain embodiments, the ligand is, but not limited to, L-cysteine, D-cysteine, or a cysteine derivative. In certain embodiments, the cysteine derivative is N-isobutyryl-L-cysteine (L-NIBC), N-isobutyryl-D-cysteine (D-NIBC), N-acetyl-L-cysteine (L-NAC), or N-acetyl-D-cysteine (D-NAC).

In certain embodiments, the ligand is, but not limited to, a cysteine-containing oligopeptide and its derivatives. In certain embodiments, the cysteine-containing oligopeptide is a cysteine-containing dipeptide. In certain embodiments, the cysteine-containing dipeptide is L(D)-cysteine-L(D)-arginine dipeptide (CR), L(D)-arginine-L(D)-cysteine dipeptide (RC), or L(D)-cysteine-L-histidine dipeptide (CH). In certain embodiments, the cysteine-containing oligopeptide is a cysteine-containing tripeptide. In certain embodiments, the cysteine-containing tripeptide is glycine-L(D)-cysteine-L(D)-arginine tripeptide (GCR), L(D)-proline-L(D)-cysteine-L(D)-arginine tripeptide (PCR), or L(D)-glutathione (GSH). In certain embodiments, the cysteine-containing oligopeptide is a cysteine-containing tetrapeptide. In certain embodiments, the cysteine-containing tetrapeptide is glycine-L(D)-serine-L(D)-cysteine-L(D)-arginine tetrapeptide (GSCR) or glycine-L(D)-cysteine-L(D)-serine-L(D)-arginine tetrapeptide (GCSR). In certain embodiments, the cysteine-containing oligopeptide is a cysteine-containing pentapeptide. In certain embodiments, the cysteine-containing pentapeptide is Cysteine-Aspartic acid-Glutamic acid-Valine-Aspartic acid (CDEVD), or Aspartic acid-Glutamic acid-Valine-Aspartic acid-Cysteine (DEVDC).

In certain embodiments, the ligand is a thiol-containing compound. In certain embodiments, thiol-containing compound is 1-[(2S)-2-methyl-3-thiol-1-oxopropyl]-L(D)-proline, thioglycollic acid, mercaptoethanol, thiophenol, D-3-trolovol, dodecyl mercaptan, 2-aminoethanethiol (CSH), 3-mercaptopropionic acid (VIPA), or 4-mercaptobenoic acid (p-MBA).

The present invention provides a pharmaceutical composition for the treatment of cerebral ischemic stroke. In certain embodiments, the subject is human. In certain embodiments, the subject is a pet animal such as a dog.

In certain embodiments, the pharmaceutical composition comprises a ligand-bound AuC as disclosed above and a pharmaceutically acceptable excipient. In certain embodiments, the excipient is phosphate-buffered solution, or physiological saline.

The present invention provides a use of the above disclosed ligand-bound AuCs for manufacturing a medication for the treatment of cerebral ischemic stroke.

The present invention provides a use of the above disclosed ligand-bound AuCs for treating a subject with cerebral ischemic stroke, or a method for treating a subject with cerebral ischemic stroke using the above disclosed ligand-bound AuCs. In certain embodiments, the method for treatment comprises administering a pharmaceutically effective amount of ligand-bound AuCs to the subject. The pharmaceutically effective amount can be ascertained by routine in vivo studies. In certain embodiments, the pharmaceutically effective amount of ligand-bound AuCs is a dosage of at least 0.001 mg/kg/day, 0.005 mg/kg/day, 0.01 mg/kg/day, 0.05 mg/kg/day, 0.1 mg/kg/day, 0.5 mg/kg/day, 1 mg/kg/day, 2 mg/kg/day, 3 mg/kg/day, 4 mg/kg/day, 5 mg/kg/day, 6 mg/kg/day, 7 mg/kg/day, 8 mg/kg/day, 9 mg/kg/day, 10 mg/kg/day, 15 mg/kg/day, 20 mg/kg/day, 30 mg/kg/day, 40 mg/kg/day, 50 mg/kg/day, 60 mg/kg/day, 70 mg/kg/day, 80 mg/kg/day, or 100 mg/kg/day.

The following examples are provided for the sole purpose of illustrating the principles of the present invention; they are by no means intended to limit the scope of the present invention.

### EMBODIMENTS

### Embodiment 1. Preparation of Ligand-Bound AuCs

1.1 Dissolving HAuCl₄ in methanol, water, ethanol, n-propanol, or ethyl acetate to get a solution A in which the concentration of HAuCl₄ is 0.01˜0.03M;

1.2 Dissolving a ligand in a solvent to get a solution B in which the concentration of the ligand is 0.01˜0.18M; the ligand includes, but not limited to, L-cysteine, D-cysteine and other cysteine derivatives such as N-isobutyryl-L-cysteine (L-NIBC), N-isobutyryl-D-cysteine (D-NIBC), N-acetyl-L-cysteine (L-NAC), and N-acetyl-D-cysteine (D-NAC), cysteine-containing oligopeptides and their derivatives including, but not limited to, dipeptides, tripeptide, tetrapeptide, pentapeptide, and other peptides containing cysteine, such as L(D)-cysteine-L(D)-arginine dipeptide (CR), L(D)-arginine-L(D)-cysteine dipeptide (RC), L(D)-cysteine L(D)-histidine (CH), glycine-L(D)-cysteine-L(D)-arginine tripeptide (GCR), L(D)-proline-L(D)-cysteine-L(D)-arginine tripeptide (PCR), L(D)-glutathione (GSH), glycine-L(D)-serine-L(D)-cysteine-L(D)-arginine tetrapeptide (GSCR), glycine-L(D)-cysteine-L(D)-serine-L(D)-arginine tetrapeptide (GCSR), Cysteine-Aspartic acid-Glutamic acid-Valine-Aspartic acid pentapeptide (CDEVD) and Aspartic acid-Glutamic acid-Valine-Aspartic acid-Cysteine pentapeptide (DEVDC), and other thiol-containing compounds, such as one or more of 1-[(2S)-2-methyl-3-thiol-1-oxopropyl]-L(D)-proline, thioglycollic acid, mercaptoethanol, thiophenol, D-3-trolovol, dodecyl mercaptan, 2-aminoethanethiol (CSH), 3-mercaptopropionic acid (MPA), and 4-mercaptobenoic acid (p-MBA); the solvent is one or more of methanol, ethyl acetate, water, ethanol, n-propanol, pentane, formic acid, acetic acid, diethyl ether, acetone, anisole, 1-propanol, 2-propanol, 1-butanol, 2-butanol, pentanol, butyl acetate, tributyl methyl ether, isopropyl acetate, dimethyl sulfoxide, ethyl formate, isobutyl acetate, methyl acetate, 2-methyl-1-propanol and propyl acetate;

1.3 Mixing solution A and solution B so that the mole ratio between HAuCl₄ and ligand is 1:(0.01˜100), stirring them in an ice bath for 0.1˜48 h, adding 0.025˜0.8M NaBH₄ water, ethanol or methanol solution, continuing to stir in an ice water bath and react for 0.1˜12 h. The mole ratio between NaBH₄ and ligand is 1:(0.01˜100);

1.4 Using MWCO 3K˜30K ultrafiltration tubes to centrifuge the reaction solution at 8000˜17500 r/min by gradient for 10˜100 min after the reaction ends to obtain ligand-bound AuCs precipitate in different average particle sizes. The aperture of the filtration membranes for ultrafiltration tubes of different MWCOs directly decides the size of ligand-bound AuCs that can pass the membranes. This step may be optionally omitted;

1.5 Dissolving the ligand-bound AuCs precipitate in different average particle sizes obtained in step (1.4) in water, putting it in a dialysis bag and dialyzing it in water at room temperature for 1˜7 days;

1.6 Freeze-drying ligand-bound AuCs for 12˜24 h after dialysis to obtain a powdery or flocculant substance, i.e., ligand-bound AuCs.

As detected, the particle size of the powdery or flocculant substance obtained by the foregoing method is smaller than 3 nm (distributed in 0.5-2.6 nm in general). No obvious absorption peak at 520 nm. It is determined that the obtained powder or floc is ligand-bound AuCs.

### Embodiment 2. Preparation and Characterization of AuCs Bound with Different Ligands

2.1 Preparation of L-NIBC-bound AuCs, i.e. L-NIBC-AuCs

Taking ligand L-NIBC for example, the preparation and confirmation of AuCs bound with ligand L-NIBC are detailed.

2.1.1 Weigh 1.00 g of HAuCl₄ and dissolve it in 100 mL of methanol to obtain a 0.03M solution A;

2.1.2 Weigh 0.57 g of L-NIBC and dissolve it in 100 mL of glacial acetic acid (acetic acid) to obtain a 0.03M solution B;

2.1.3 Measure 1 mL of solution A, mix it with 0.5 mL, 1 mL, 2 mL, 3 mL, 4 mL, or 5 mL of solution B respectively (i.e. the mole ratio between HAuCl₄ and L-NIBC is 1:0.5, 1:1, 1:2, 1:3, 1:4, 1:5 respectively), react in an ice bath under stirring for 2 h, quickly add 1 mL of freshly prepared 0.03M (prepared by weighing 11.3 mg of NaBH₄ and dissolving it in 10 mL of ethanol) NaBH₄ ethanol solution when the solution turns colorless from bright yellow, continue the reaction for 30 min after the solution turns dark brown, and add 10 mL of acetone to terminate the reaction.

2.1.4 After the reaction, the reaction solution is subjected to gradient centrifugation to obtain L-NIBC-AuCs powder with different particle sizes. Specific method: After the reaction is completed, the reaction solution is transferred to an ultrafiltration tube with MWCO of 30K and a volume of 50 mL, and centrifuged at 10000 r/min for 20 min, and the retentate in the inner tube is dissolved in ultrapure water to obtain powder with a particle size of about 2.6 nm. Then, the mixed solution in the outer tube is transferred to an ultrafiltration tube with a volume of 50 mL and MWCO of 10K, and centrifuged at 13,000 r/min for 30 min. The retentate in the inner tube is dissolved in ultrapure water to obtain powder with a particle size of about 1.8 nm. Then the mixed solution in the outer tube is transferred to an ultrafiltration tube with a volume of 50 mL and MWCO of 3K, and centrifuged at 17,500 r/min for 40 min. The retentate in the inner tube is dissolved in ultrapure water to obtain powder with a particle size of about 1.1 nm.

2.1.5 Precipitate the powder in three different particle sizes obtained by gradient centrifugation, remove the solvent respectively, blow the crude product dry with N2, dissolve it in 5 mL of ultrapure water, put it in a dialysis bag (MWCO is 3 KDa), put the dialysis bag in 2 L of ultrapure water, change water every other day, dialyze it for 7 days, freeze-dry it and keep it for future use.

2.2 Characterization of L-NIBC-AuCs

Characterization experiment was conducted for the powder obtained above (L-NIBC-AuCs). Meanwhile, ligand L-NIBC-modified gold nanoparticles (L-NIBC-AuNPs) are used as control. The method for preparing gold nanoparticles with ligand being L-NIBC refers to the reference (W. Yan, L. Xu, C. Xu, W. Ma, H. Kuang, L. Wang and N. A. Kotov, Journal of the American Chemical Society 2012, 134, 15114; X. Yuan, B. Zhang, Z. Luo, Q. Yao, D. T. Leong, N. Yan and J. Xie, Angewandte Chemie International Edition 2014, 53, 4623).

2.2.1 Observation of the Morphology by Transmission Electron Microscope (TEM)

The test powders (L-NIBC-AuCs sample and L-NIBC-AuNPs sample) were dissolved in ultrapure water to 2 mg/L as samples, and then test samples were prepared by hanging drop method. More specifically, 5 μL of the samples were dripped on an ultrathin carbon film, volatized naturally till the water drop disappeared, and then observe the morphology of the samples by JEM-2100F STEM/EDS field emission high-resolution TEM.

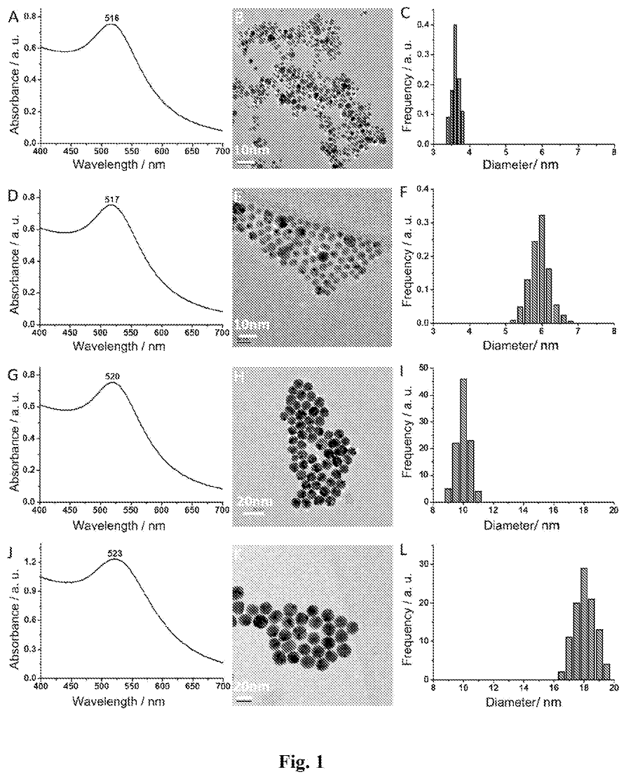

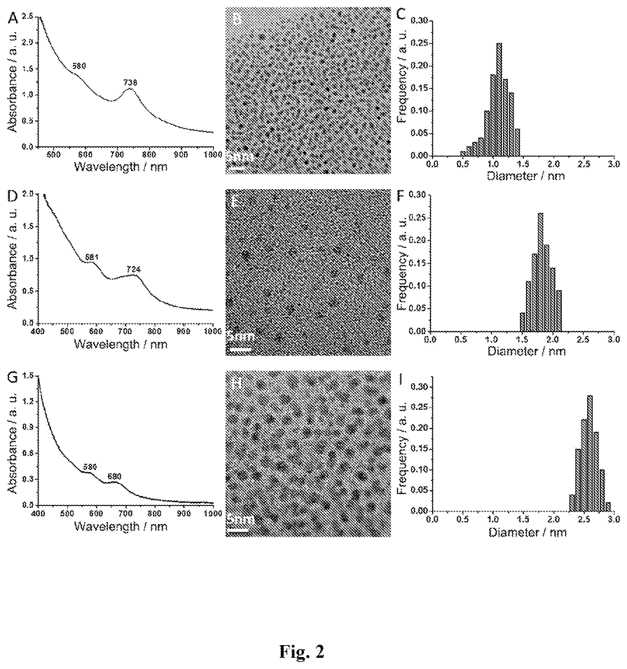

The four TEM images of L-NIBC-AuNPs are shown in panels B, E, H, and K of FIG. 1; the three TEM images of L-NIBC-AuCs are shown in panels B, E, and H of FIG. 2.

The images in FIG. 2 indicate that each of L-NIBC-AuCs samples has a uniform particle size and good dispersibility, and the average diameter of L-NIBC-AuCs (refer to the diameter of gold core) is 1.1 nm, 1.8 nm and 2.6 nm respectively, in good accordance with the results in panels C, F and I of FIG. 2. In comparison, L-NIBC-AuNPs samples have a larger particle size. Their average diameter (refer to the diameter of gold core) is 3.6 nm, 6.0 nm, 10.1 nm and 18.2 nm respectively, in good accordance with the results in panels C, F, I and L of FIG. 1.

2.2.2 Ultraviolet (UV)-Visible (vis) Absorption Spectra

The test powders (L-NIBC-AuCs sample and L-NIBC-AuNPs sample) were dissolved in ultrapure water till the concentration was 10 mg·L⁻¹, and the UV-vis absorption spectra were measured at room temperature. The scanning range was 190-1100 nm, the sample cell was a standard quartz cuvette with an optical path of 1 cm, and the reference cell was filled with ultrapure water.

The UV-vis absorption spectra of the four L-NIBC-AuNPs samples with different sizes are shown in panels A, D, G and J of FIG. 1, and the statistical distribution of particle size is shown in panels C, F, I and L of FIG. 1; the UV-vis absorption spectra of three L-NIBC-AuCs samples with different sizes are shown in panels A, D and G of FIG. 2, and the statistical distribution of particle size is shown in panels C, F and I of FIG. 2.

FIG. 1 indicates that due to the surface plasmon effect, L-NIBC-AuNPs had an absorption peak at about 520 nm. The position of the absorption peak is relevant with particle size. When the particle size is 3.6 nm, the UV absorption peak appears at 516 nm; when the particle size is 6.0 nm, the UV absorption peak appears at 517 nm; when the particle size is 10.1 nm, the UV absorption peak appears at 520 nm, and when the particle size is 18.2 nm, the absorption peak appears at 523 nm. None of the four samples has any absorption peak above 560 nm.

FIG. 2 indicates that in the UV absorption spectra of three L-NIBC-AuCs samples with different particle sizes, the surface plasmon effect absorption peak at 520 nm disappeared, and two obvious absorption peaks appeared above 560 nm and the positions of the absorption peaks varied slightly with the particle sizes of AuCs. This is because AuCs exhibit molecule-like properties due to the collapse of the face-centered cubic structure, which leads to the discontinuity of the density of states of AuCs, the energy level splitting, the disappearance of plasmon resonance effect and the appearance of a new absorption peak in the long-wave direction. It could be concluded that the three powder samples in different particle sizes obtained above are all ligand-bound AuCs.

2.2.3 Fourier Transform Infrared Spectroscopy

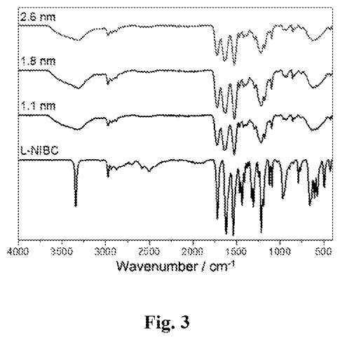

Infrared spectra were measured on a VERTEX80V Fourier transform infrared spectrometer manufactured by Bruker in a solid powder high vacuum total reflection mode. The scanning range is 4000-400 cm⁻¹ and the number of scans is 64. Taking L-NIBC-AuCs samples for example, the test samples were L-NIBC-AuCs dry powder with three different particle sizes and the control sample was pure L-NIBC powder. The results are shown in FIG. 3.

FIG. 3 shows the infrared spectrum of L-NIBC-AuCs with different particle sizes. Compared with pure L-NIBC (the curve at the bottom), the S—H stretching vibrations of L-NIBC-AuCs with different particle sizes all disappeared completely at 2500-2600 cm⁻¹, while other characteristic peaks of L-NIBC were still observed, proving that L-NIBC molecules were successfully bound to the surface of AuCs via Au—S bond. The figure also shows that the infrared spectrum of the ligand-bound AuCs is irrelevant with its size.

AuCs bound with other ligands were prepared by a method similar to the above method, except that the solvent of solution B, the feed ratio between HAuCl₄ and ligand, the reaction time and the amount of NaBH₄ added were slightly adjusted. For example: when L-cysteine, D-cysteine, N-isobutyryl-L-cysteine (L-NIBC) or N-isobutyryl-D-cysteine (D-NIBC) is used as the ligand, acetic acid is selected as the solvent; when dipeptide CR, dipeptide RC or 1-[(2S)-2-methyl-3-thiol-1-oxopropyl]-L-proline is used as the ligand, water is selected as the solvent, and so on and so forth; other steps are similar, so no further details are provided herein.

The present invention prepared and obtained a series of ligand-bound AuCs by the foregoing method. The ligands and the parameters of the preparation process are shown in Table 1.

TABLE 1
Preparation parameters of AuCs bound with different ligands in the present invention

| | | Parameter | | | | | | |
|---|---|---|---|---|---|---|---|---|
| | | | | | | | Time of | |
| | | | | | | | reaction | |
| | | | | Time of | | | in an ice | |
| | | | | reaction in | | | bath | |
| | | | | an ice bath | | Mole | under | |
| | | | | under | | ratio | stirring | |
| | | | Feed ratio | stirring | | between | after | |
| | | | between | before | | HAuCl₄ | addition | |
| | | Solvent used | HAuCl₄ and | addition | | and | of | |
| | Ligand | for solution B | ligand | of NaBH₄ | | NaBH₄ | NaBH₄ | |
| 1 | L-cysteine | Acetic acid | 1:3 | 2 | h | 1:2 | 0.5 | h |
| 2 | D-cysteine | Acetic acid | 1:3 | 2 | h | 1:2 | 0.5 | h |
| 3 | N-acetyl-L-cysteine | Ethanol | 1:4 | 1 | h | 1:1 | 0.5 | h |
| 4 | N-acetyl-D-cysteine | Ethanol | 1:4 | 1 | h | 1:1 | 0.5 | h |
| 5 | L-NIBC | Water | 1:4 | 0.5 | h | 1:2 | 0.5 | h |
| 6 | D-NIBC | Water | 1:4 | 0.5 | h | 1:2 | 0.5 | h |
| 7 | Other cysteine | Soluble | 1:(0.1~100) | 0.5 h~24 h | | 1:(0.1~100) | 0.1~24 | h |
| | derivatives | solvent | | | | | | |
| 8 | CR | Water | 1:4 | 22 | h | 2:1 | 0.5 | h |
| 9 | RC | Water | 1:4 | 20 | h | 2:1 | 0.5 | h |
| 10 | HC | Water | 1:3 | 12 | h | 1:2 | 2 | h |
| 11 | CH | Ethanol | 1:4 | 16 | h | 1:3 | 3 | h |
| 12 | GSH | Water | 1:2 | 12 | h | 1:1 | 3 | h |
| 13 | KCP | Water | 1:3 | 15 | h | 1:2 | 1 | h |
| 14 | PCR | Water | 1:4 | 16 | h | 1:3 | 2 | h |
| 15 | GSCR | Water | 1:4 | 16 | h | 1:3 | 1.5 | h |
| 16 | GCSR | Water | 1:3 | 12 | h | 1:2 | 2 | h |
| 17 | CDEVD | Water | 1:7 | 1 | h | 1:0.1 | 0.5 | h |
| 18 | DEVDC | Water | 1:7 | 1 | h | 1:0.1 | 0.5 | h |
| 19 | Other oligopeptides | Soluble | 1:(0.1~100) | 0.5 h~24 h | | 1:(0.1~100) | 0.1~24 | h |
| | containing cysteine | solvent | | | | | | |
| 20 | 1-[(2S)-2-methyl-3- | Water | 1:8 | 2 | h | 1:7 | 1 | h |
| | thiol-1-oxopropyl]-L- | | | | | | | |
| | proline | | | | | | | |
| 21 | Mercaptoethanol | Ethanol | 1:2 | 2 | h | 1:1 | 1 | h |
| 22 | Thioglycollic acid | Acetic acid | 1:2 | 2 | h | 1:1 | 1 | h |
| 23 | Thiophenol | Ethanol | 1:5 | 5 | h | 1:1 | 1 | h |
| 24 | D-3-trolovol | Water | 1:2 | 2 | h | 1:1 | 1 | h |
| 25 | N-(2- | Water | 1:2 | 2 | h | 1:1 | 1 | h |
| | mercaptopropionyl)- | | | | | | | |
| | glycine | | | | | | | |
| 26 | Dodecyl mercaptan | Methanol | 1:5 | 5 | h | 1:1 | 1 | h |
| 27 | 2-aminoethanethiol | Water | 1:5 | 2 | h | 8:1 | 0.5 | h |
| | (CSH) | | | | | | | |
| 28 | 3-mercaptopropionic | Water | 1:2 | 1 | h | 5:1 | 0.5 | h |
| | acid (MPA) | | | | | | | |
| 29 | 4-mercaptobenoic | Water | 1:6 | 0.5 | h | 3:1 | 2 | h |

| | acid (p-MBA) | | | | | | |
|---|---|---|---|---|---|---|---|
| 30 | Other compounds | Soluble | 1:(0.01~100) | 0.5 h~24 h | 1:(0.1~100) | 0.1~24 | h |

| | |
|---|---|
| containing thiol | solvent |

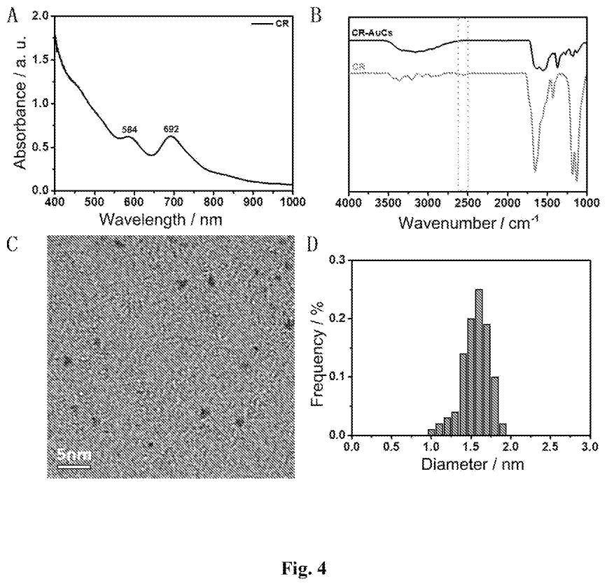

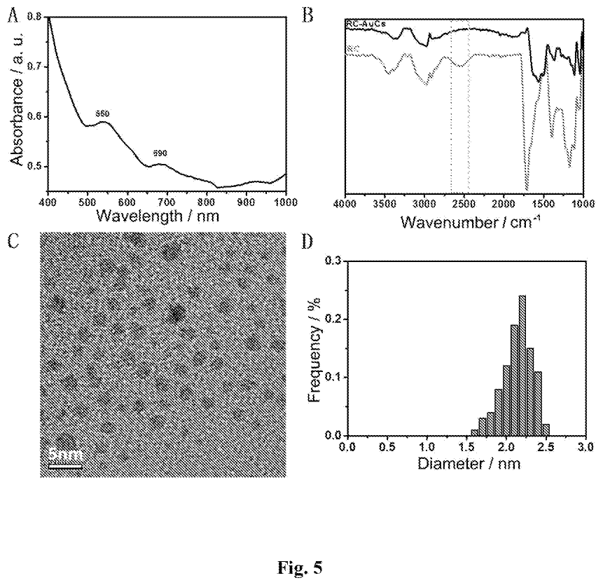

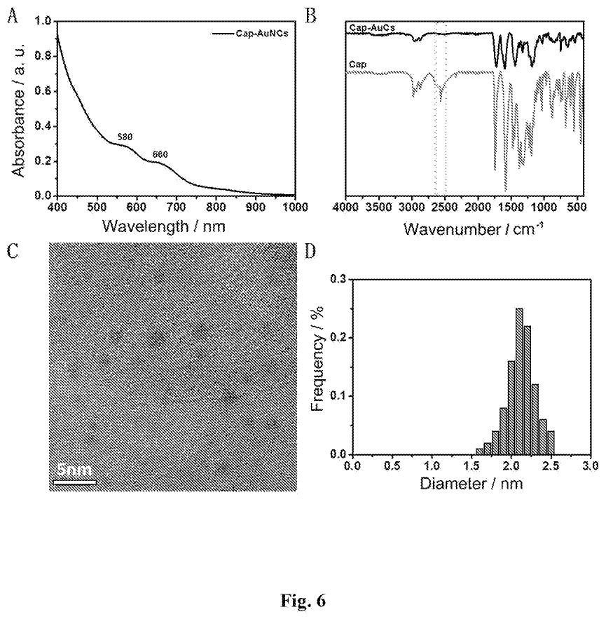

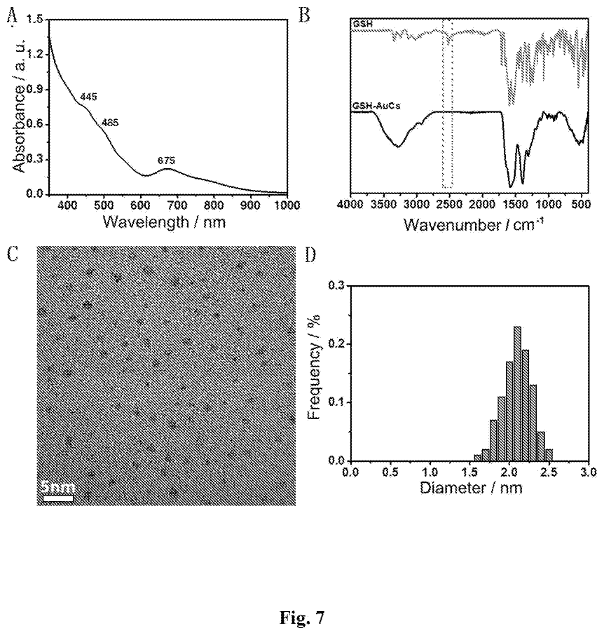

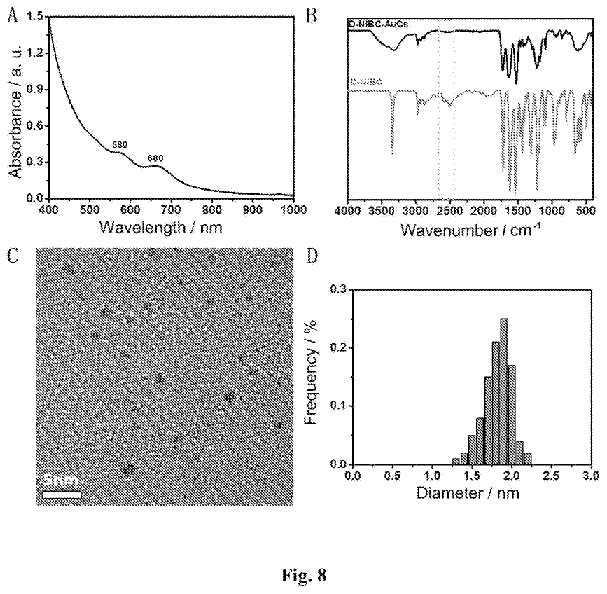

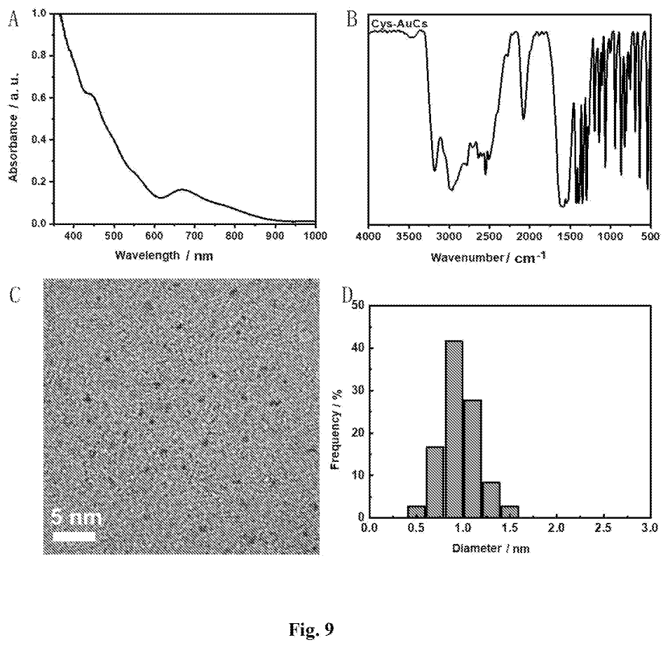

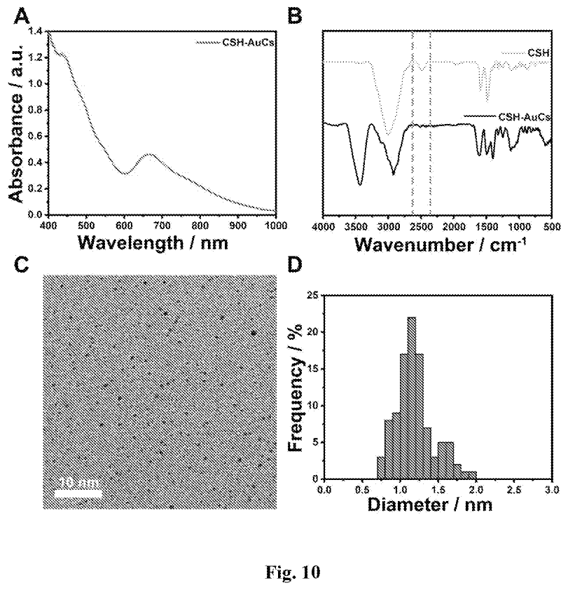

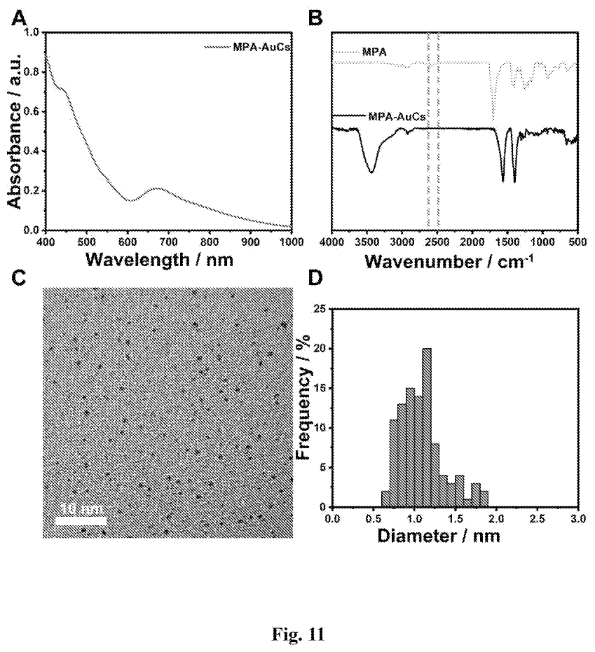

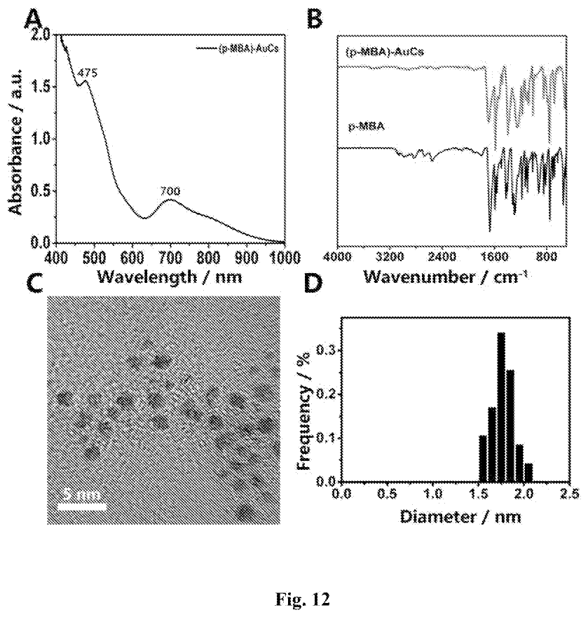

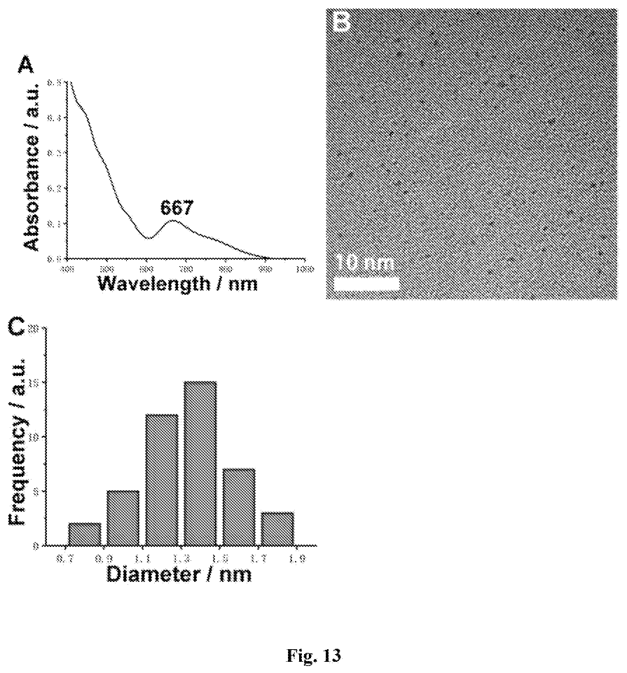

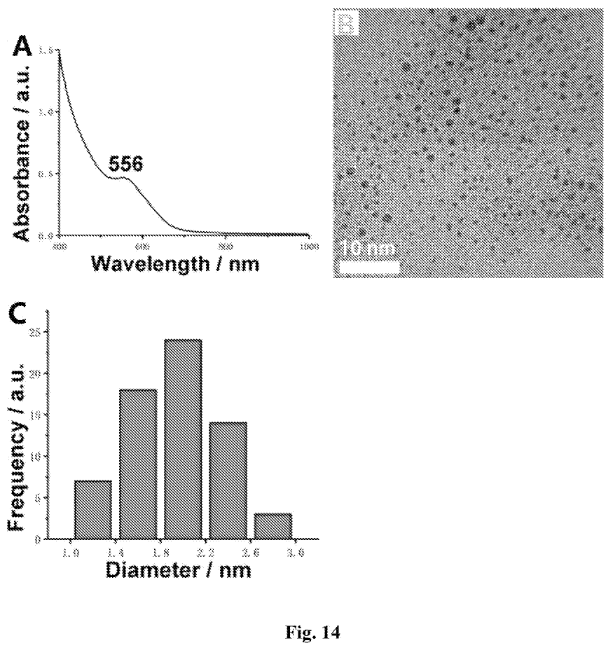

The samples listed in Table 1 are confirmed by the foregoing methods. The characteristics of eleven (11) different ligand-bound AuCs are shown in FIG. 4 (CR-AuCs), in FIG. 5 (RC-AuCs), in FIG. 6 (Cap-AuCs) (Cap denotes 1-[(2S)-2-methyl-3-thiol-1-oxopropyl]-L-proline), in FIG. 7 (GSH-AuCs), in FIG. 8 (D-NIBC-AuCs), in FIG. 9 (L-Cys-AuCs), in FIG. 10 (CSH-AuCs), in FIG. 11 (MPA-AuCs), in FIG. 12 (p-MBA-AuCs), in FIG. 13 (CDEVD-AuCs), and in FIG. 14 (DEVDC-AuCs). FIG. 4-FIG. 12 show UV spectra (panel A), infrared spectra (panel B), TEM images (panel C), and particle size distribution (panel D). FIGS. 13 and 14 show UV spectra (panel A), TEM images (panel B), and particle size distribution (panel C).

The results indicate that the diameters of AuCs bound with different ligands obtained from Table 1 are all smaller than 3 nm. Ultraviolet spectra also show disappearance of peak at 520±20 nm, and appearance of absorption peak in other positions. The position of the absorption peak could vary with ligands and particle sizes as well as structures. In certain situations, there is no special absorption peak, mainly due to the formation of AuCs mixtures with different particles sizes and structures or certain special AuCs that moves the position of absorption peak beyond the range of UV-vis spectrum. Meanwhile, Fourier transform infrared spectra also show the disappearance of ligand thiol infrared absorption peak (between the dotted lines in panel B of FIGS. 4-8), while other infrared characteristic peaks are all retained, suggesting that all ligand molecules have been successfully bound to gold atoms to form ligand-bound AuCs, and the present invention has successfully obtained AuCs bound with the ligands listed in Table 1.

### Embodiment 3. Cerebral Ischemic Stroke Animal Model Experiments

3.1 Testing Samples

Gold Clusters:
A1: ligand L-NIBC-bound gold clusters (L-NIBC-AuCs), size distribution in the range of 0.5-3.0 nm;
A2: ligand L-cysteine-bound gold clusters (L-Cys-AuCs), size distribution in the range of 0.5-3.0 nm;
A3: ligand N-acetyl-L-cysteine-bound gold clusters (L-NAC-AuCs), size distribution in the range of 0.5-3.0 nm; and
A4: ligand DEVDC-bound gold clusters (DEVDC-AuCs), size distribution in the range of 0.5-3.0 nm.

Gold Nanoparticles:
B1: L-NIBC-bound gold nanoparticles (L-NIBC-AuNPs), size distribution range of 6.1±1.5 nm; and
B2: L-NAC-bound gold nanoparticles (L-NAC-AuNPs), size distribution range of 9.0±2.4 nm.

All testing samples were prepared following the above described method with slight modification, and their quality was characterized using the above described methods.

3.2 Experimental Protocols

3.2.1 Establishment of Rat Middle Cerebral Artery Occlusion (MCAO) Model and Administration of Test Substances

Male SPF grade Sprague Dawley (SD) rats (220-260 g) were purchased from Shanghai Shrek Experimental Animal Co., Ltd. All rats were acclimatized to the environment for 7 days prior to the experiments. Rats were randomly divided into 14 groups (n=10), including sham operation group, model control group, low (2 mg/kg rat body weight) and high-dose group (10 mg/kg rat body weight) of gold cluster drugs A1, A2, A3 and A4, and low (2 mg/kg rat body weight) and high-dose group (10 mg/kg rat body weight) of gold nanoparticle B1 and B2. On the day of the experiments, the rats were anesthetized with 10% chloral hydrate (350 mg/kg body weight). The right common carotid artery, internal carotid artery and external carotid artery were exposed through the midline incision. The suture was inserted into the internal carotid artery (ICA) 18 mm±0.5 mm through the external carotid artery (ECA), until the MCA regional blood supply was blocked, resulting in cerebral infarction. After 1.5 h, the suture was withdrawn to the entrance of ECA for reperfusion. The basic cerebral blood flow (CBF) before operation and after embolization were measured by flow meter. The animals whose CBF decreased continuously (rCBF≥70%) were considered to be successful models of middle cerebral artery occlusion (MCAO). After reperfusion, the rats were injected intraperitoneally with drugs or solvents (normal saline) at 0 h, 24 h, 48 h and 72 h respectively. The neurological behavior scores were evaluated at 0 h, 24 h, 48 h, 72 h and 96 h. The experiment was terminated at 96 h after operation. Brain collection and TTC staining were performed after euthanasia. Images of brain slices were taken and the percentage of cerebral infarction area was calculated.

3.2.2 Neurological Behavior Score

0 point: no difference from normal rats; 1 point: right front paw extension is not straight, head to the opposite side; 2 points: walking discontinuous circles in the open space; 3 points: walking continuous circles in the open space; 4 points, unconscious walking, collapse to one side; 5 points: death.

3.2.3 Infarct Area (TTC Staining)

The rats were euthanized by carbon dioxide inhalation. The brains were taken and put into the brain trough for coronal section (2 mm). Staining was with 2% TTC in dark at room temperature. After taking photos, the infarct area was analyzed by ImageJ. The percentage of infarct area (%)=(contralateral hemisphere area−(ipsilateral hemisphere area−infarction area))/contralateral hemisphere area×100%.

3.2.4 Statistical Analysis

Statistical analysis was performed by Graph Pad Prism Software 7.0 (CA, US). The data were expressed as mean±standard error, and the statistical analysis was performed by Dunnett test. P<0.05 denotes statistically significant.

3.3 Results

3.3.1 Cerebral Blood Flow in the Cerebral Ischemic Region

More than 70% decrease of rat cerebral blood flows (reduction cerebral blood flow, rCBF≥70%) indicates successful establishment of MACO model. Except for the sham operation group, all remaining groups had rCBF more than 70%, with an average of about 80%, demonstrating successful establishment of MCAO model.

3.3.2 Effects of Each Drug on Rat Neurological Behavior

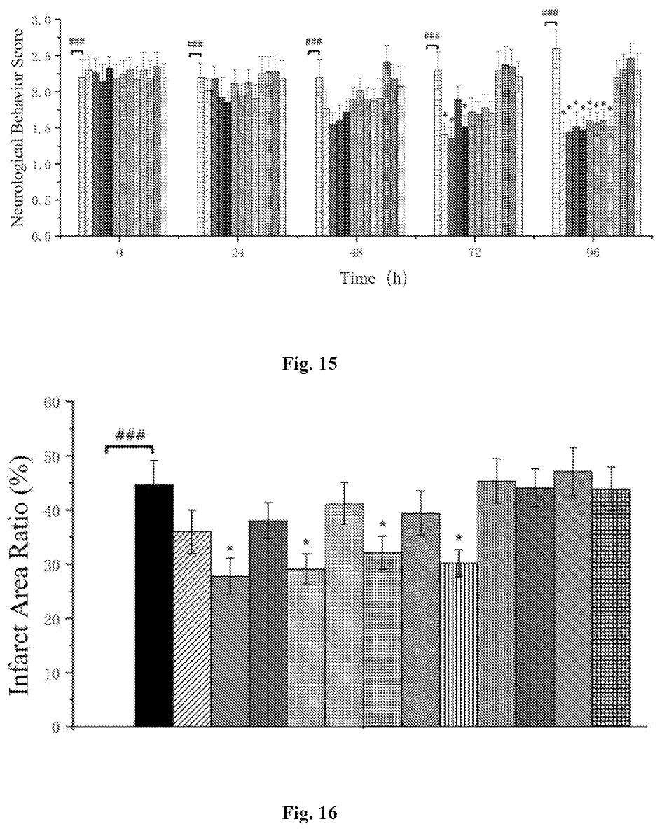

FIG. 15 shows the neurological behavior scores of rats in each group (in the histogram of each time point, from left to right are sham operation group (blank), model control group, A1 low-dose group, A1 high-dose group, A2 low-dose group, A2 high-dose group, A3 low-dose group, A3 high-dose group, A4 low-dose group, A4 high-dose group, B1 low-dose group, B1 high-dose group, B2 low-dose group and B2 high-dose group). The rats in the sham operation group had normal neurological behavior, and the behavior score was 0; the rats in the model control group showed severe behavioral functional defects at 0 h, 24 h, 48 h, 72 h and 96 h after operation (compared with the sham operation group, P<0.001, ###). Compared with the model control group, the neurological behavior scores of A1, A2, A3, A4 low-dose groups and high-dose groups had no significant improvement at 24 hours after operation. At 48 h post operation, the neurological behavior scores of A1, A2, A3 and A4 low-dose groups and high-dose groups began to decline, but there was no statistical difference (compared with the model control group, P>0.05). At 72 h post operation, the neurological behavior scores of the four drugs were further decreased, among which A1 low-dose group, A1 high-dose group and A2 high-dose group showed significant differences (compared with model control group, P<0.05, *). At 96 h post operation, there were significant differences for all the low-dose groups and the high-dose groups of the four drugs (compared with the model control group, P<0.05, *). These results suggest that all four gold cluster drugs can significantly improve the neurological behavior deficits induced by ischemic stroke, and the effect is dose-dependent to a certain extent.

Compared with the model control group, the low and high dose groups of gold nanoparticles B1 and B2 did not significantly improve the neurological behavior scores of MACO model rats at 24 h, 48 h, 72 h and 96 h after operation, indicating that gold nanoparticles could not significantly improve the behavioral disorders caused by cerebral ischemic stroke.

3.3.3.3 Effect of Each Drug on Cerebral Infarction Areas of MACO Model Rats

FIG. 16 shows the percentage of cerebral infarction area of rats in each group (in the histogram, from left to right are sham operation group (blank), model control group, A1 low-dose group, A1 high-dose group, A2 low-dose group, A2 high-dose group, A3 low-dose group, A3 high-dose group, A4 low-dose group, A4 high-dose group, B1 low-dose group, B1 high-dose group, B2 low-dose group and B2 high-dose group). In the sham operation group, the brain tissue was normal and no infarction occurred; the infarct area was 0%. The infarct area of the model control group was 44.7%±4.5% (P<0.001, ###). Compared with the model control group, the percentages of cerebral infarction areas in A1, A2, A3, A4 low and high-dose groups were evidently decreased, but there was no significant difference in the low-dose groups, while significant difference was found in the high-dose groups (compared with model control group, P<0.05, *). Taking A1 as an example, the infarct area of the low-dose group decreased from 44.7±4.5% to 36.0±4.0% (compared with model control group, P>0.05), while that of high-dose group decreased to 27.8±3.4% (compared with model control group, P<0.05, *).

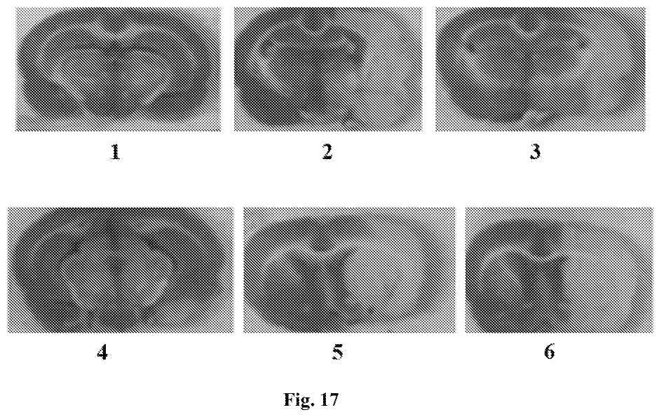

FIG. 17 presents the exemplary images of TTC staining brain tissues of MCAO rats after administration of the gold clusters drugs represented by A1 and gold nanoparticles represented by B1. In FIG. 17, (1) sham operation group; (2) model control group; (3) A1 low-dose group; (4) A1 high-dose group; (5) B1 low-dose group; (6) B1 high-dose group. As can be seen from FIG. 17, the rats in the sham operation group did not have cerebral infarction, while the model control group had a large cerebral infarction (white part on the right). The area of cerebral infarction after low-dose administration of A1 drug was reduced (the white part on the right side was reduced), while the area of cerebral infarction was significantly reduced by high-dose administration of A1 drug (the white part on the right side was greatly reduced), while the low-dose and high-dose administration of B1 had no effect on the area of cerebral infarction (the white part on the right side had no reduction). A2, A3 and A4 showed similar effect to A1 in reducing infarct area, while B2 was similar to B1 with no reduction of infarct area.

Other ligand-bound AuCs also have the similar effects on treating cerebral ischemic stroke, while their effects vary to certain extents. They would not be described in detail here.

### Embodiment 4. Cerebral Hemorrhagic Stroke Animal Model Experiments

4.1 Regent

Ligand L-cysteine-bound gold clusters (L-Cys-AuCs), size distribution in the range of 0.5-3.0 nm; L-NIBC-bound gold nanoparticles (L-NIBC-AuNPs), size distribution range of 6.1±1.5 nm.

4.2 Experimental Protocol and Results

Rats are anesthetized and placed in a stereotaxic frame. On day 0, Type VII collagenase was stereotactically injected into the right striatum (coordinates: 0.0 mm rostral and 3.0 mm lateral to bregma, 5.5 mm below the skull) at 0.4 μl/min over 5 min. The test drugs are administered i.p. from day 0 to day 4 at a dosage of 10 mg/kg rat weight. Locomotion is measured on day 3. Rats are sacrificed on day 4 for analysis and histochemistry staining. The tested AuCs drug and gold nanoparticles showed similar results on cerebral hemorrhagic stroke, indicating no apparent therapeutical effects.

While the present invention has been described with reference to particular embodiments, it will be understood that the embodiments are illustrative and that the invention scope is not so limited. Alternative embodiments of the present invention will become apparent to those having ordinary skill in the art to which the present invention pertains. Such alternate embodiments are considered to be encompassed within the scope of the present invention. Accordingly, the scope of the present invention is defined by the appended claims and is supported by the foregoing description.

### REFERENCES

Amani H, Mostafavi E, Mahmoud Reza Alebouyeh M R, Arzaghi H, Akbarzadeh A, Pazoki-Toroudi H, Webster T J. Would Colloidal Gold Nanocarriers Present An Effective Diagnosis Or Treatment For Ischemic Stroke? Int J Nanomedicine. 2019 Oct. 7; 14:8013-8031.
Zheng Y, Wu Y, Liu Y, Guo Z, Bai T, Zhou P, Wu J, Yang Q, Liu Z, Lu X. Intrinsic Effects of Gold Nanoparticles on Oxygen-Glucose Deprivation/Reperfusion Injury in Rat Cortical Neurons. Neurochem Res. 2019 July; 44(7):1549-1566.

1. Use of a ligand-bound gold cluster to treat cerebral ischemic stroke in a subject, wherein the ligand-bound gold cluster comprises:
a gold core; and
a ligand bound to the gold core.

2. The use of claim 1, wherein the gold core has a diameter in the range of 0.5-3 nm.

3. The use of claim 1, wherein the gold core has a diameter in the range of 0.5-2.6 nm.

4. The use of claim 1, wherein the ligand is one selected from the group consisting of L-cysteine and its derivatives, D-cysteine and its derivatives, cysteine-containing oligopeptides and their derivatives, and other thiol-containing compounds.

5. The use of claim 4, wherein the L-cysteine and its derivatives are selected from the group consisting of L-cysteine, N-isobutyryl-L-cysteine (L-NIBC), and N-acetyl-L-cysteine (L-NAC), and wherein the D-cysteine and its derivatives are selected from the group consisting of D-cysteine, N-isobutyryl-D-cysteine (D-NIBC), and N-acetyl-D-cysteine (D-NAC).

6. The use of claim 4, wherein the cysteine-containing oligopeptides and their derivatives are cysteine-containing dipeptides, wherein the cysteine-containing dipeptides are selected from the group consisting of L(D)-cysteine-L(D)-arginine dipeptide (CR), L(D)-arginine-L(D)-cysteine dipeptide (RC), L(D)-histidine-L(D)-cysteine dipeptide (HC), and L(D)-cysteine-L(D)-histidine dipeptide (CH).

7. The use of claim 4, wherein the cysteine-containing oligopeptides and their derivatives are cysteine-containing tripeptides, wherein the cysteine-containing tripeptides are selected from the group consisting of glycine-L(D)-cysteine-L(D)-arginine tripeptide (GCR), L(D)-proline-L(D)-cysteine-L(D)-arginine tripeptide (PCR), L(D)-lysine-L(D)-cysteine-L(D)-proline tripeptide (KCP), and L(D)-glutathione (GSH).

8. The use of claim 4, wherein the cysteine-containing oligopeptides and their derivatives are cysteine-containing tetrapeptides, wherein the cysteine-containing tetrapeptides are selected from the group consisting of glycine-L(D)-serine-L(D)-cysteine-L(D)-arginine tetrapeptide (GSCR), and glycine-L(D)-cysteine-L(D)-serine-L(D)-arginine tetrapeptide (GCSR).

9. The use of claim 4, wherein the cysteine-containing oligopeptides and their derivatives are cysteine-containing pentapeptide, wherein the cysteine-containing pentapeptides are selected from the group consisting of Cysteine-Aspartic acid-Glutamic acid-Valine-Aspartic acid (CDEVD) and Aspartic acid-Glutamic acid-Valine-Aspartic acid -Cysteine (DEVDC).

10. The use of claim 4, wherein the other thiol-containing compounds are selected from the group consisting of 1-[(2S)-2-methyl-3-thiol-1-oxopropyl]-L(D)-proline, thioglycollic acid, mercaptoethanol, thiophenol, D-3-trolovol, N-(2-mercaptopropionyl)-glycine, dodecyl mercaptan, 2-aminoethanethiol (CSH), 3-mercaptopropionic acid (MPA), and 4-mercaptobenoic acid (p-MBA).

11. Use of a ligand-bound gold cluster (AuC) for manufacture of a medicament for the treatment of cerebral ischemic stroke in a subject, wherein the ligand-bound gold cluster comprises:
a gold core; and
a ligand bound to the gold core.

12. The use of claim 11, wherein the gold core has a diameter in the range of 0.5-3 nm.

13. The use of claim 11, wherein the gold core has a diameter in the range of 0.5-2.6 nm.

14. The use of claim 11, wherein the ligand is one selected from the group consisting of L-cysteine and its derivatives, D-cysteine and its derivatives, cysteine-containing oligopeptides and their derivatives, and other thiol-containing compounds.

15. The use of claim 14, wherein the L-cysteine and its derivatives are selected from the group consisting of L-cysteine, N-isobutyryl-L-cysteine (L-NIBC), and N-acetyl-L-cysteine (L-NAC), and wherein the D-cysteine and its derivatives are selected from the group consisting of D-cysteine, N-isobutyryl-D-cysteine (D-NIBC), and N-acetyl-D-cysteine (D-NAC).

16. The use of claim 14, wherein the cysteine-containing oligopeptides and their derivatives are cysteine-containing dipeptides, wherein the cysteine-containing dipeptides are selected from the group consisting of L(D)-cysteine-L(D)-arginine dipeptide (CR), L(D)-arginine-L(D)-cysteine dipeptide (RC), L(D)-histidine-L(D)-cysteine dipeptide (HC), and L(D)-cysteine-L(D)-histidine dipeptide (CH).

17. The use of claim 14, wherein the cysteine-containing oligopeptides and their derivatives are cysteine-containing tripeptides, wherein the cysteine-containing tripeptides are selected from the group consisting of glycine-(D)L-cysteine-L(D)-arginine tripeptide (GCR), L(D)-proline-L(D)-cysteine-L(D)-arginine tripeptide (PCR), L(D)-lysine-L(D)-cysteine-L(D)-proline tripeptide (KCP), and L(D)-glutathione (GSH).

18. The use of claim 14, wherein the cysteine-containing oligopeptides and their derivatives are cysteine-containing tetrapeptides, wherein the cysteine-containing tetrapeptides are selected from the group consisting of glycine-L(D)-serine-L(D)-cysteine-L(D)-arginine tetrapeptide (GSCR), and glycine-L(D)-cysteine-L(D)-serine-L(D)-arginine tetrapeptide (GCSR).

19. The use of claim 14, wherein the cysteine-containing oligopeptides and their derivatives are cysteine-containing pentapeptide, wherein the cysteine-containing pentapeptides are selected from the group consisting of Cysteine-Aspartic acid-Glutamic acid-Valine-Aspartic acid (CDEVD) and Aspartic acid-Glutamic acid-Valine-Aspartic acid -Cysteine (DEVDC).

20. The use of claim 14, wherein the other thiol-containing compounds are selected from the group consisting of 1-[(2S)-2-methyl-3-thiol-1-oxopropyl]-L(D)-proline, 24 hioglycolic acid, mercaptoethanol, thiophenol, D-3-trolovol, N-(2-mercaptopropionyl)-glycine, dodecyl mercaptan, 2-aminoethanethiol (CSH), 3-mercaptopropionic acid (MPA), and 4-mercaptobenoic acid (p-MBA).
